(19) United States
(12) Patent Application Publication
(10) Pub. No.: US 2014/0378840 A1
(43) Pub. Date: Dec. 25, 2014

(54) ASSESSMENT DEVICE AND ASSESSMENT METHOD

(71) Applicant: ENDOFLOW, LLC, Baltimore, MD (US)

(72) Inventor: Eric KRAUT, Baltimore, MD (US)

(21) Appl. No.: 14/302,657

(22) Filed: Jun. 12, 2014

Related U.S. Application Data
(60) Provisional application No. 61/837,267, filed on Jun. 20, 2013.

(51) Int. Cl. A61B 8/08; A61B 8/06

(52) U.S. Cl. CPC A61B 8/488; A61B 8/065; A61B 8/0883
USPC 600/453

(57) ABSTRACT

An assessment device and assessment method are provided. The assessment device includes a tube, a flexible tip secured to the tube, and a rotatable sensor section proximal to the flexible tip, the rotatable sensor section including a Doppler transducer and a phased array arranged and disposed to provide a Doppler assessment data point corresponding to an assessment angle of the Doppler transducer. The rotatable sensor section is rotatable between a first assessment angle and a second assessment angle. The assessment method includes providing an assessment device including the rotatable sensor section, inserting the rotatable sensor section, positioning the rotatable sensor section with respect to a target assessment area, rotating the rotatable sensor section through a range of assessment angles between a first assessment angle and a second assessment angle, and obtaining the Doppler assessment data point with the rotatable sensor section.

### CROSS-REFERENCE TO RELATED APPLICATION

This application claims the benefit of and priority to U.S. Provisional Patent Application Ser. No. 61/837,267 entitled “Nasogastric Fluid Optimization Probe” filed on Jun. 20, 2013, which is hereby incorporated by reference.

### FIELD OF THE INVENTION

The present invention is directed toward an assessment device and assessment method. intravascular volume assessment device. More specifically, the present invention is directed to an intravascular volume assessment device and method of assessing intravascular volume.

### BACKGROUND OF THE INVENTION

An accurate assessment of a patient's volume status is extremely important to survival, yet difficult to obtain in many clinical scenarios including sepsis, acute respiratory distress syndrome (ARDS), acute lung injury (ALI), hemorrhagic shock, cardiogenic shock, and situations where there are massive fluid shifts such as open abdominal surgery. Patient volume status assessments have been made using many different measures including urine output, arterial line variation, Vigileo monitor, central venous lines, and Swan Ganz catheters. All of these measures are indirect assessments of the left ventricular end diastolic volume (LVEDV). All of the known assessment techniques relating to indirect measurement of the LVEDV have inaccuracies related to assumptions made to determine the volume.

For example, known indirect methods for determining LVEDV include central venous pressure (CVP) and pulmonary artery (PA) wedge pressure, which rely on a measured pressure from the right side of the heart to estimate the pressure of the left side of the heart. The estimated pressure of the left side of the heart is assumed to correspond to the volume of the left side of the heart. However, the pressure of the left side of the heart does not correspond to the volume of the left side under various conditions, such as valvular disease, sepsis, ARDS, and other pathologic conditions that change the compliance, size and shape of the heart. The standard measurement based on the right ventricle may inaccurately reflect the left ventricular end diastolic volume by a factor of up to two. (Kraut, J Trauma. 1997) In another example, urine output can be altered by conditions unrelated to volume status such as, but not limited to, acute tubular necrosis, end-stage renal disease, intra-abdominal hypertension, or combinations thereof.

The most accurate method of determining LVEDV is by directly measuring it. A direct assessment of the LVEDV can be made by looking at the left ventricle of the heart with ultrasound as a single point in time using either transthoracic or transesophageal echocardiography. Often, direct measurement of LVEDV in critically ill patients results in only a single snapshot of a dynamic condition. The single snapshot in time may fail to capture vital information for diagnosis and treatment. In addition, techniques, such as computed tomography (CT), magnetic resonance imaging (MRI), and nuclear imaging, require moving the patient which can be traumatic and dangerous. Currently there are no continuous, real time assessments of the LVEDV that may be followed by medical staff over a period of days. Furthermore, methods such as transesophageal echo require trained specialists to perform the test each time.

Intravascular fluid imaging devices and methods that do not suffer from one or more of the above drawbacks would be desirable in the art.

### BRIEF DESCRIPTION OF THE INVENTION

In one exemplary embodiment, an assessment device includes a tube, a flexible tip secured to the tube, and a rotatable sensor section proximal to the flexible tip, the rotatable sensor section including a Doppler transducer and a phased array. The Doppler transducer and the phased array are arranged and disposed to provide a Doppler assessment data point corresponding to an assessment angle of the Doppler transducer, and the rotatable sensor section is rotatable between a first assessment angle and a second assessment angle.

In another exemplary embodiment, an assessment device includes a tube including a first end and a second end, a handle secured to the first end, the handle including a positioning mechanism, a flexible tip secured to the second end, and a rotatable sensor section proximal to the flexible tip, the rotatable sensor section comprising a Doppler transducer and a phased array. The positioning mechanism positions the rotatable sensor section with respect to a target assessment area, the Doppler transducer and the phased array are arranged and disposed to provide a Doppler assessment data point corresponding to an assessment angle of the Doppler transducer, the rotatable sensor section is rotatable between a first assessment angle and a second assessment angle, and the Doppler assessment data point includes one or both of a measured value of intravascular volume and a two-dimensional image of the target assessment area.

In another exemplary embodiment, an assessment method includes providing an assessment device including a tube, a flexible tip secured to the tube, and a rotatable sensor section proximal to the flexible tip, the rotatable sensor section including a Doppler transducer and a phased array arranged and disposed to provide a Doppler assessment data point corresponding to an assessment angle of the Doppler transducer, inserting the rotatable sensor section, positioning the rotatable sensor section with respect to a target assessment area, rotating the rotatable sensor section through a range of assessment angles between a first assessment angle and a second assessment angle, and obtaining the Doppler assessment data point with the rotatable sensor section.

Other features and advantages of the present invention will be apparent from the following more detailed description of the preferred embodiment, taken in conjunction with the accompanying drawings which illustrate, by way of example, the principles of the invention.

### BRIEF DESCRIPTION OF THE DRAWINGS

FIG. 1 shows a perspective view of an assessment device according to an embodiment of the disclosure.

FIG. 2 shows an enhanced view of an assessment device according to an embodiment of the disclosure.

FIG. 3 is a flow chart of an assessment method according to an embodiment of the disclosure.

FIG. 4 shows an assessment device inserted in a transgastric short axis view.

Wherever possible, the same reference numbers will be used throughout the drawings to represent the same parts.

### DETAILED DESCRIPTION OF THE INVENTION

Provided are an assessment device and assessment method. Embodiments of the present disclosure, in comparison to assessment devices and assessment methods not using one or more of the features disclosed herein, provide continuous real-time intravascular volumes, increase assessment efficiency, increase measurement accuracy, decrease patient movement, permit left ventricular volume measurements in disease states, or a combination thereof.

An assessment system according to the present disclosure includes an assessment apparatus, an assessment device 100 coupled to the assessment apparatus, and assessment algorithms/software for assessing flow and/or volume from a plurality of assessment device 100 positions. In one embodiment, the assessment apparatus includes an ultrasound system, such as, but not limited to, a 64-channel ultrasound machine that provides at least B-mode imaging, M-mode imaging, pulsed Doppler, and color flow Doppler. In another embodiment, the assessment apparatus includes any suitable probe board, such as, but not limited to, a probe board having a 260-pin zero insertion force probe connector, a 64 to 192 multiplexer, a Tx connector, and an Rx connector. In another embodiment, the assessment apparatus includes any suitable receiver, such as, but not limited to, a 64-channel receiver with an Rx connector, two 32-channel receiver modules, two autofocus 32-channel receive beamformers, ultrasound control and mid-proc functions, a universal serial bus (USB) 2.0, and a clock generator. In a further embodiment, the assessment apparatus includes any suitable transmitter, such as, but not limited to, a 64-channel transmitter with a Tx connector, 5 level pulsers, and a 64-channel transmit beamformer. A power supply for the assessment system includes, but is not limited to, a passive backplane, an HV power module +/−100v, an HV power module +/−50v, LV power supplies, or a combination thereof.

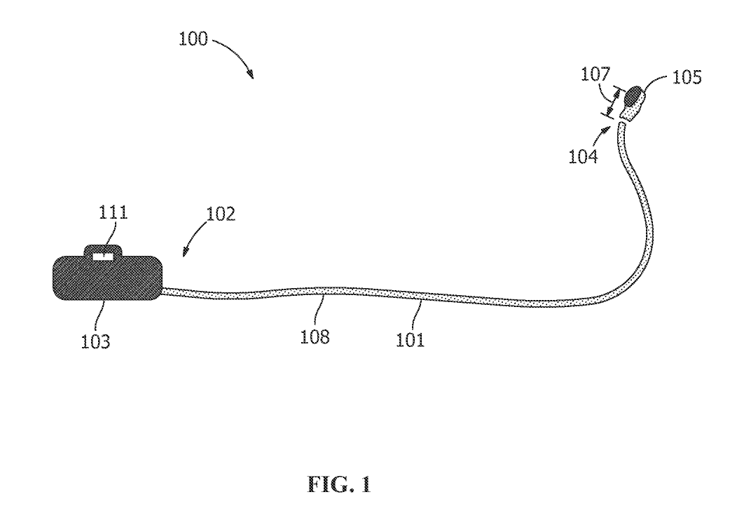

Referring to FIG. 1, in one embodiment, the assessment device 100 includes a tube 101 having a first end 102, a handle 103 secured to the first end 102, an internal channel 108, a second end 104, a flexible tip 105 secured to the second end 104, a first row of crystals, a second row of crystals, and a rotatable sensor section 107. The tube 101 includes any suitable tube for insertion into an individual's body. In one embodiment, the tube 101 includes a pliant material suitable for positioning within or through the esophagus, such as, but not limited to, rubber, polyurethane, silicone, polyvinyl chloride (PVC), or a combination thereof. In another embodiment, the internal channel 108 of the tube 101 is divided into multiple channels for housing wires and/or cables, and providing suction. In one example, the tube 101 includes a nasogastric tube (NGT) for insertion through an individual's nose. In another example, the tube 101 is a nasoesophageal tube, which may be similar to an NGT, but is not fully inserted into the stomach.

The tube 101 includes any suitable diameter for insertion through the individual's nose. Suitable diameters include, but are not limited to, up to about 10 mm (30 Fr), between about 4 mm (12 Fr) and about 10 mm (30 Fr), between about 5 mm (15 Fr) and about 9 mm (27 Fr), between about 6 mm (18 Fr) and about 8 mm (24 Fr), about 6 mm (18 Fr), or any combination, sub-combination, range, or sub-range thereof. In another embodiment, the tube 101 includes any suitable length for positioning the second end 104 within an individual to provide a Doppler assessment data point corresponding to two dimensional images or volumes such as, but not limited to, intraventricular volumes. Suitable lengths of the tube 101 include, but are not limited to, between about 91 cm (36 inches) to about 153 cm (60 inches), between about 106 cm (42 inches) to about 138 cm (54 inches), about 122 cm (48 inches), or any combination, sub-combination, range, or sub-range thereof. For example, in one embodiment, the tube 101 is a dual lumen NGT including a sump port to provide suction of air, the NGT having a diameter of 18 Fr and a length of about 122 cm (e.g. Salem Sump™, commercially available from Covidien, Mansfield, Mass., Ref. No. 8888266148).

The handle 103 secured to the first end 102 includes a positioning mechanism 111. The positioning mechanism 111 on the handle 103 positions the rotatable sensor section 107 with respect to a target assessment area. In one embodiment, positioning the rotatable sensor section 107 includes bending the tube 101 and the flexible tip 105 without bending the rotatable sensor section 107 proximal to the flexible tip 105. The positioning mechanism 111 includes any suitable mechanism for positioning the rotatable sensor section 107 with respect to the target assessment area, including, but not limited to, a trigger, a button, a switch, an electronic control, or a combination thereof. In another embodiment, the positioning mechanism 111 is coupled to the flexible tip 105 by a coupling means such as, but not limited to, wires extending through the tube 101.

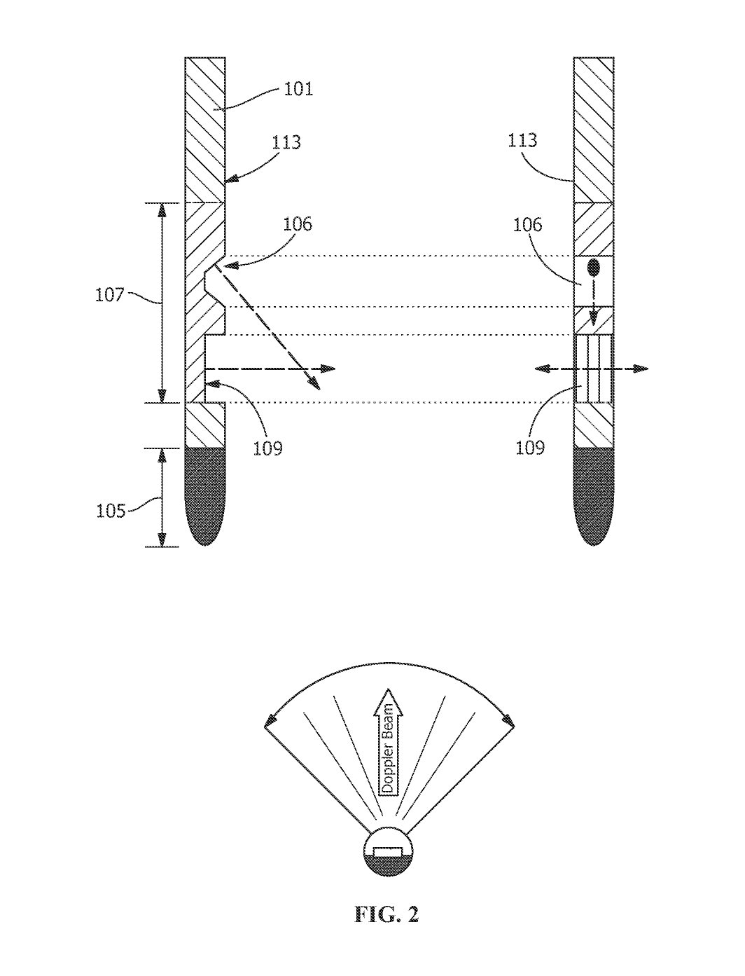

Referring to FIG. 2, in one embodiment, the rotatable sensor section 107 includes any suitable Doppler transducer 106 for providing a Doppler assessment data point corresponding to measurement of intravascular volumes. Suitable Doppler transducers include, but are not limited to, an ultrasound probe, a side viewing Doppler, a pulsed wave Doppler, or a continuous wave Doppler including two rows of piezoelectric crystals. In another embodiment, the rotatable sensor section 107 includes the first row of crystals. In a further embodiment, the first row of crystals includes, but is not limited to, a phased array 109. The Doppler transducer 106 and the phased array 109 are arranged and disposed to provide a Doppler assessment data point corresponding to an assessment angle. In a further embodiment, the rotatable sensor section 107 is rotatable between a first assessment angle and a second assessment angle. The first assessment angle corresponds to a first viewable angle of the target assessment area, and the second assessment angle corresponds to a second viewable angle of the target assessment area. For example, in one embodiment, the first viewable angle is an initial viewable angle and the second viewable angle is a final viewable angle. In other embodiments, combinations of viewable angles between the first and second assessment angles are utilized to provide desired data. In one embodiment, the rotatable sensor section 107 provides continuous Doppler assessment data throughout a range of assessment angles between the first assessment angle and the second assessment angle. In a further embodiment, the rotatable sensor section 107 is rotatable independent of the tube 101.

“Doppler assessment”, as utilized herein, means a measured or calculated value of a condition, such as a volume, flow rate, physical makeup or other medically relevant property of a patient. Doppler assessment may include, but is not limited to one or more Doppler assessment data points, such as, but not limited to, a volume measurement or two dimensional image, or may include a calculated or analyzed value calculated or formed from a plurality of Doppler assessment data points, such as, but not limited to, a three dimensional image or calculated ejection volume of a target assessment area.

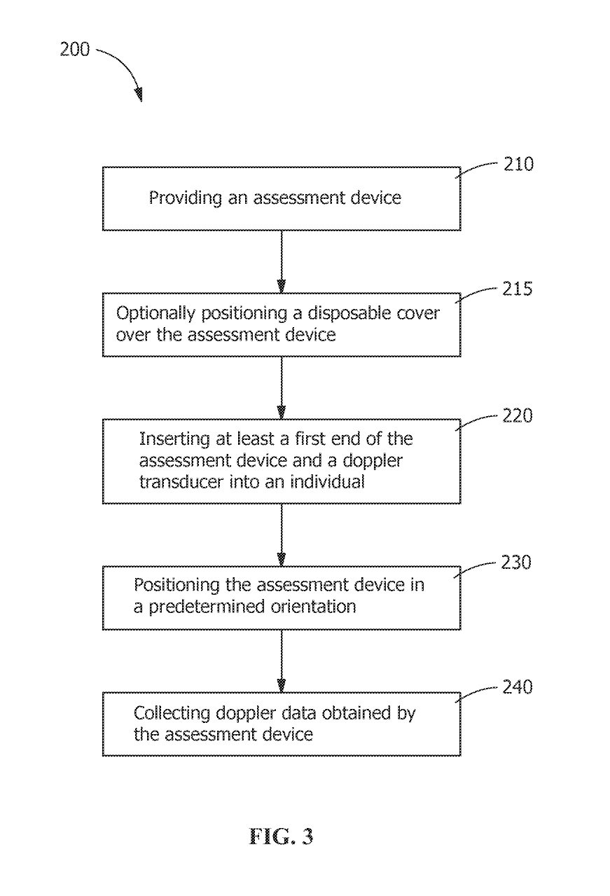

Referring to FIG. 3, in one embodiment, an assessment method 200 for obtaining Doppler data includes providing the assessment device 100 (step 210), optionally positioning a disposable cover 113 over the assessment device 100 (step 215), inserting the rotatable sensor section 107 (step 220), positioning the assessment device 100 with respect to the target assessment area (step 230), rotating the rotatable sensor section 107 to assessment angles between the first assessment angle and the second assessment angle, and obtaining the Doppler assessment data point with the assessment device 100 (step 240). The rotating may result in discrete assessment within the range between the first assessment angle and the second assessment angle, or the rotating may result in continuous measurement at assessment angles through a range between the first assessment angle and the second assessment angle.

The rotatable sensory section 107 is inserted (step 220) by any suitable method, such as, but not limited to, through an individual's nose, or through an individual's oral cavity. During insertion (step 220) the flexible tip 105 reduces or eliminates trauma and/or inflammation of the sinuses. The flexible tip 105 includes any suitable length for reducing or eliminating trauma during insertion of the assessment device 100. Suitable lengths of the flexible tip 105 include, but are not limited to, between about 10 mm and about 20 mm, between about 12 mm and about 20 mm, between about 12 mm and about 18 mm, between about 14 mm and about 16 mm, about 15 mm, or any combination, sub-combination, range, or sub-range thereof.

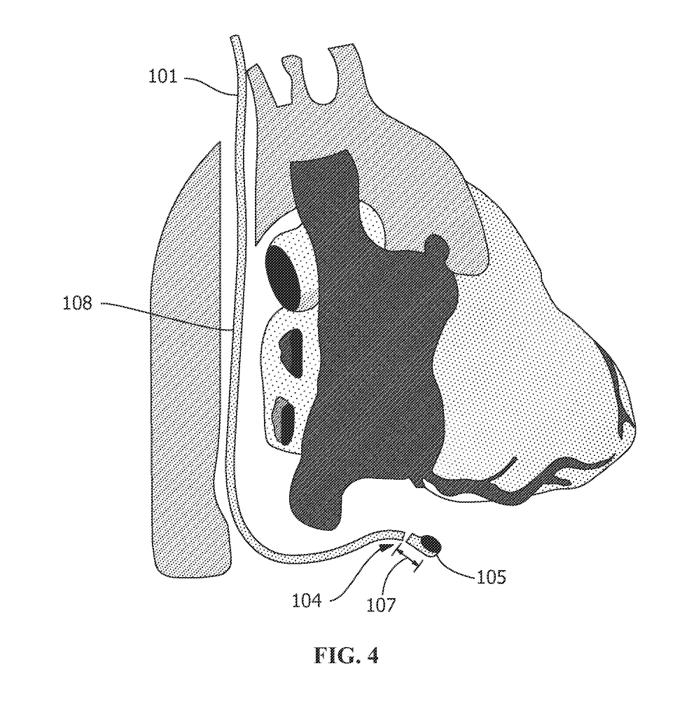

Referring to FIG. 4, in one embodiment, positioning the assessment device 100 with respect to the target assessment area (step 230) includes advancing the assessment device 100 a predetermined insertion distance corresponding to the target assessment area. The predetermined insertion distance includes any suitable distance for obtaining an echocardiogram from the rotatable sensor section 107. Suitable distances include, but are not limited to, between about 25 cm and about 50 cm from an individual's incisors, between about 28 cm and about 45 cm from the individual's incisors, between about 28 cm and about 32 cm from the individual's incisors, between about 35 cm and about 45 cm from the individual's incisors, or any combination, sub-combination, range, or sub-range thereof. In another embodiment, the tube 101 includes markings and/or indicia corresponding to various insertion distances, such as, but not limited to, markings at 5 mm intervals. Varying the predetermined insertion distance provides variations in the corresponding target assessment area.

In one embodiment, the predetermined insertion distance positions the rotatable sensor section 107 in an area adjacent the gastroesophageal junction to provide transesophageal images corresponding to the left ventricle. For example, in one embodiment, the rotatable sensor section 107 is positioned within the esophagus to obtain a Doppler assessment of the descending aorta. In another example, the predetermined insertion distance of about 32 cm from an individual's incisors (about 37 cm from the individual's nostril) corresponds to a four-chamber view of the heart. In another example, the predetermined insertion distance of between about 42 cm and about 45 cm from the individual's incisors (about 45 to about 48 cm from the individual's nostril) with full flexion and horizontal (0 degree) imaging corresponds to a modified apical four-chamber view of the heart.

In an alternate embodiment, advancing the assessment device 100 the predetermined distance includes advancing the rotatable sensor section 107, including the Doppler transducer 106, through an individual's gastroesophageal junction and into the fundus of an individual's stomach. The rotatable sensor section 107 in the fundus provides a direct view of the left ventricle of an individual's heart, which lies just above the fundus of the stomach. In one example, advancing the rotatable sensor section 107 into the individual's stomach provides a transgastric short axis view of the left ventricle. Advancement of the rotatable sensor section 107 into the fundus is confirmed by insufflation and auscultation through the assessment device 100. After confirming advancement of the rotatable sensor section 107, the assessment device 100 is bent about 90 degrees and then pulled back. Once the assessment device 100 has been positioned, a fan-shaped beam from the rotatable sensor section 107 is aimed upward about 90 degrees relative to an axis of the esophagus and the stomach to obtain the short axis view of the left ventricle. In one embodiment, the rotatable sensor section 107 is rotated to obtain different planes or cuts through the heart, for example. In another embodiment, the different planes or cuts are then be used to calculate volume.

The insertion of the assessment device 100 (step 220) is performed by any suitable individual, such as, but not limited to, a nurse or a physician. The assessment device 100 inserted (step 220) as described herein, permits assessment of intravascular volume without moving the individual to a different location. For example, the individual's intravascular volume may be assessed in the intensive care unit, in the operating room, in the emergency room, while the individual is in a regular floor bed, or any other suitable location. In another embodiment, the assessment device 100 provides monitoring of an individual without risks associated with invasive monitors, risks such as, but not limited to, pneumothorax, hemothorax, air embolus, arrhythmia, thrombosis, and death.

In one embodiment, positioning the assessment device 100 with respect to the target assessment area (step 230) includes verifying a position of the rotatable sensor section 107. In one example, verifying the position of the rotatable sensor section 107 includes obtaining an image with the second row of crystals, and displaying the image on a display device. The second row of crystals is either positioned within the rotatable sensory section 107, or adjacent to the rotatable sensor section 107. In another example, verifying the position of the rotatable sensor section 107 includes, but is not limited to, obtaining a chest x-ray, providing air through the suction channel and listening over the stomach, or a combination thereof.

In another embodiment, after insertion of the assessment device 100 (step 220), positioning of the assessment device 100 (step 230) includes flexion of the assessment device 100 and/or rotation of the rotatable sensor section 107. Flexion of the assessment device 100 includes activation of the positioning mechanism 111 to adjust the rotatable sensor section 107 with respect to the target assessment area. Adjusting the rotatable sensor section 107 may include modifying an orientation of the rotatable sensor section 107 relative to the target assessment area by bending the tube 101 and/or the flexible tip 105 without bending the rotatable sensor section 107. Together, the tube 101 and the flexible tip 105 bend up to about 120 degrees without kinking the internal channel 108. The bending of the tube 101 and/or the flexible tip 105 provides various views from within the individual. The various views correspond to differing views of one target assessment area, or views of differing target assessment areas, such as, but not limited to, the four-chamber view of the heart, the modified apical four-chamber view of the heart, a descending aorta view, a left ventricle view, and a left ventricular outflow tract view.

In one embodiment, during positioning of the assessment device 100 (step 230) the tube 101 remains rotationally fixed while the rotatable sensor section 107 is rotated in any suitable increment, such as, but not limited to, up to about 15 degrees, up to about 30 degrees, up to about 60 degrees, or a combination thereof to allow measurement of Doppler assessment data points at the corresponding angles. Measurement by the assessment device 100 at an assessment angle is not limited to a single data point, but may include multiple data points, including combinations of different types of measured data. In another embodiment, after positioning the assessment device 100 (step 230), the rotatable sensor section 107 is rotated through the range of assessment angles between the first assessment angle and the second assessment angle to provide continuous Doppler assessment from the continuous measurement of the Doppler assessment data points. The assessment device 100 obtains assessment data points, which includes, but is not limited to, two-dimensional images of the target assessment area, fluid velocities (flow), volume, or a combination thereof. In a further embodiment, a plurality of Doppler assessment data points including two-dimensional images are reconstructed to form a Doppler assessment including one or more three-dimensional images. For example, in one embodiment, the assessment device 100 obtains up to six two-dimensional transesophageal images, rotating the rotatable sensor section 107 between about 15 degrees and about 30 degrees after each image. After obtaining the images, the six two-dimensional transesophageal images are reconstructed to form a three-dimensional image of a left ventricle without relying on geometric assumptions. In one embodiment, the Doppler assessment data points obtained by the assessment device 100 include, but are not limited to, two-dimensional images of the target assessment area, fluid velocities (flow), volume, or a combination thereof. For example, the data points correspond to left ventricular end diastolic volume and left ventricular end systolic volume.

In one embodiment, the internal channel 108 provides suction and/or infusion through the assessment device 100 to reduce an amount of air interference, decompress the individual's stomach, and/or provide enteral access for tube feeding (e.g., for critically ill patients). For example, applying continuous suction of between about 30 mmHg and about 40 mmHg to the internal channel 108 reduces the amount of air interference and decompresses the individuals stomach. In another embodiment, the size, the suction, and/or the infusion permits maintaining the assessment device 100 within the individual for extended periods of time. For example, in one embodiment, the assessment device 100 may be continuously maintained within the individual for up to 7 days, between 12 hours and 7 days, between 24 hours and 5 days, up to about 48 hours, or any combination, sub-combination, range, or sub-range thereof. In a further embodiment, the section is continuously applied at a pressure including, but not limited to, between about 30 mmHg and about 40 mmHg. Maintaining the assessment device 100 within the individual permits either repeated collection of single data points, or continuous collection of data points obtained by the assessment device 100 (step 240) positioned within the individual (step 230).

In one embodiment, the collected data points obtained by the assessment device 100 are analyzed with software including pre-programmed algorithms for providing measurements such as, but not limited to, left ventricular end diastolic volume, left ventricular end systolic volume, ejection fraction, cardiac output, and stroke volume. In one embodiment, the continuously collected data points are analyzed in real-time to provide the measurements on a beat-to-beat basis. In another embodiment, the collected data points and/or the Doppler assessment corresponding to the Doppler assessment data point are digitized and displayed for interpretation on the display device, such as, but not limited to, a monitor attached to the assessment device 100, a remote monitor (e.g., wirelessly, through electronic medical records), a hand-held device (e.g., smart-phone, tablet), or a combination thereof. In a further embodiment, the collected data points and/or the Doppler assessment corresponding to the Doppler assessment data point are displayed on the display device for interpretation during procedures such as, but not limited to, giving fluid, withholding fluid, forced diuresis, administering medications (e.g., medications to improve cardiac contractility), or a combination thereof. For example, the collected data points and/or the Doppler assessment corresponding to the Doppler assessment data point displayed on the display device may be provided to guide fluid infusion, guide resuscitation, prevent dehydration, prevent pulmonary edema, indentify poor contractility, manage fluid, manage cardiac function, or a combination thereof.

When the positioning of the assessment device 100 (step 230) corresponds to one of the target assessment areas for measuring left ventricle diameters, the rotatable sensor section 107 obtains non-forshortened images of the left ventricular cavity from an esophageal view without the difficulty experienced by other devices. The target assessment areas for measuring left ventricle diameters include, but are not limited to, midesophageal and transgastric views. Obtaining the non-forshortened images permits quantitation of the left ventricular volume from the esophageal view, while reducing or eliminating any differences between a transesophageal echo (TEE) and a transthoracic echo (TTE).

The software including the pre-programmed algorithms analyzes the collected data points using acoustic quantification methods including, but not limited to, two-dimensional quantitiation and determines the Doppler assessment. For example, in one embodiment, the two-dimensional quantitation of the left ventricular volume is performed from the two-dimensional transesophageal images obtained by the assessment device 100. In an alternate embodiment, the software focuses on a region of interest in the four-chamber view and the transgastric short axis view corresponding to the left ventricle to analyze the collected data points and provide a digital readout display of the left ventricle volumes.

In one embodiment, the two-dimensional quantitation method for determining the Doppler assessment includes a disk algorithm, such as, but not limited to, a biplane method of disks (modified Simpson's rule). The biplane method of disks provides quantitation based upon a principle that a total left ventricular volume is calculated from the summation of a stack of elliptical discs. A height of each disk is calculated as a fraction such as, but not limited to, 1/20 of a long axis based on the longer of the two lengths of the two-chamber view and the four-chamber view. A cross-sectional area of each disc is based on two diameters obtained from the two-chamber view and the four-chamber views.

Other methods of left ventricular quantitation for determining the Doppler assessment include, but are not limited to, area-length method, linear measurement estimations, two-dimensional estimation, three-dimensional echocardiograph, or combinations thereof.

The area-length method is an alternative, for example, when apical endocardial definition is not clear. In the area-length method, the left ventricle is assumed to be bullet shaped. A mid-left ventricle cross-sectional area is computed by planimetry. Length is taken from a midpoint of the annulus to the apex in the four-chamber view. Repeated measurements are then taken at end diastole and end systole and the following formula is used:

Volume−(5(area)(length))/6

The linear measurement estimations assume that the left ventricle can be described as a prolate ellipse. Thus the left ventricular volume is approximately iD³ where iD is a short axis diameter of the left ventricle at the tips of the mitral valve (cube formula).

The two-dimensional estimation of left ventricular volume is based on the assumption that the ventricle has the shape of a truncated ellipsoid (area length formula). The biplane method of disks (modified Simpson's rule) and a method of multiple diameters generally provide more accurate measurements than the linear measurement (M-Mode).

In one embodiment, a three-dimensional approach for determining the Doppler assessment, such as three-dimensional echocardiography using three-dimensional imaging, measures the left ventricular volume without making assumptions about the geometry of the left ventricle. The three-dimensional approach provides increased volume characterization as compared to the two-dimensional quantitation methods, and is not subject to plane positioning errors which can lead to chamber forshortening. By measuring the left ventricular volume without making assumptions about the geometry of the left ventricle, the three-dimensional approach reduces or eliminates inaccuracies resulting from the assumptions, thus providing increased accuracy.

The three-dimensional approaches are divided into two techniques, a first technique based on an offline reconstruction, and a second technique based on an online data acquisition using a matrix-array transducer known as real-time three-dimensional echo. After acquiring raw data, calculating the left ventricular volumes requires identification of endocardial borders. Using manual or semi automated algorithms, the endocardial borders are then processed using disk methods to calculate volume.

Accuracy of the data points collected by the assessment device 100 and the measurements calculated by the software including the pre-programmed algorithms is not decreased by complications such as, but not limited to, changes in compliance of the heart in disease states (e.g., ARDS, ALI, shock), valvular heart disease, or increases in intrathoracic pressure due to disease states or ventilator changes.

In one embodiment, the tube 101 provides EKG monitoring. In another embodiment, an end diastolic volume (EDV) and an end systolic volume (ESV) are continuously measured from the continuously collected data points obtained by the assessment device 100. In a further embodiment, ejection fraction (EF) is then determined by the following formula:

EF=(EDV−ESV)/EDV

Prior to determining the ejection fraction, the left ventricular EDV is defined as being measured at any suitable point in a cardiac cycle. For example, in one embodiment, the left ventricular EDV is defined at the onset of a QRS complex. In another embodiment, the left ventricular EDV is defined as a first frame after mitral valve closure. Other suitable points in the cardiac cycle include, but are not limited to, a frame in the cardiac cycle where cardiac dimension is largest (e.g., the frame following atrial contraction in sinus rhythm), or a frame preceding mitral valve opening (e.g., a time in the cardiac cycle when chamber size is smallest).

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. An assessment device, comprising:
a tube;
a flexible tip secured to the tube; and
a rotatable sensor section proximal to the flexible tip, the rotatable sensor section comprising:
a Doppler transducer; and
a phased array;
wherein the Doppler transducer and the phased array are arranged and disposed to provide a Doppler assessment data point corresponding to an assessment angle of the Doppler transducer; and
wherein the rotatable sensor section is rotatable between a first assessment angle and a second assessment angle.

2. The assessment device of claim 1, further comprising a positioning mechanism.

3. The assessment device of claim 1, wherein the rotatable sensor section provides continuous Doppler assessment throughout a range of assessment angles between the first assessment angle and the second assessment angle.

4. The assessment device of claim 1, wherein a length of the flexible tip comprises between about 10 mm and about 20 mm.

5. The assessment device of claim 1, wherein the rotatable sensor section rotates independently of the tube.

6. The assessment device of claim 1, wherein the Doppler transducer is a side viewing Doppler transducer.

7. The assessment device of claim 1, wherein the Doppler transducer further comprises a continuous wave Doppler.

8. The assessment device of claim 1, wherein the Doppler assessment data point includes a measured value of intravascular volume.

9. An assessment device, comprising:
a tube including a first end and a second end;
a handle secured to the first end, the handle including a positioning mechanism;
a flexible tip secured to the second end; and
a rotatable sensor section proximal to the flexible tip, the rotatable sensor section comprising:
a Doppler transducer; and
a phased array;
wherein the positioning mechanism positions the rotatable sensor section with respect to a target assessment area;
wherein the Doppler transducer and the phased array are arranged and disposed to provide a Doppler assessment data point;
wherein the rotatable sensor section is rotatable between a first assessment angle and a second assessment angle; and
wherein the Doppler assessment data point includes one or both of a measured value of intravascular volume and a two-dimensional image of the target assessment area.

10. An assessment method, comprising:
providing an assessment device, comprising:
a tube;
a flexible tip secured to the tube; and
a rotatable sensor section proximal to the flexible tip, the rotatable sensor section comprising a Doppler transducer and a phased array;
wherein the Doppler transducer and the phased array are arranged and disposed to provide a Doppler assessment data point corresponding to an assessment angle of the Doppler transducer;
inserting the rotatable sensor section;
positioning rotatable sensor section with respect to a target assessment area;
rotating the rotatable sensor section through a range of assessment angles between a first assessment angle and a second assessment angle; and
obtaining the Doppler assessment data point with the rotatable sensor section.

11. The method of claim 10, wherein the positioning the rotatable sensor section with respect to the target assessment area comprises advancing the rotatable sensor section to an area adjacent an individual's gastroesophageal junction.

12. The method of claim 10, wherein positioning comprises activating a positioning mechanism on a handle of the assessment device, the positioning mechanism bending the assessment device without bending the rotatable sensor section.

13. The method of claim 10, wherein rotating the rotatable sensor section further comprises providing a continuous Doppler assessment through the range of assessment angles between the first assessment angle and the second assessment angle.

14. The method of claim 10, wherein the Doppler assessment data point is selected from the group consisting of a two-dimensional image, a measured flow, a measured volume, and combinations thereof.

15. The method of claim 10, wherein a Doppler assessment is derived from an algorithm for continuously analyzing Doppler assessment data points to provide measurements selected from the group consisting of stroke volume, cardiac output, left ventricular end diastolic volume, left ventricular end systolic volume, and ejection fraction.

16. The method of claim 15, further comprising continuously analyzing the measured Doppler assessment data points in real-time to provide information during procedures selected from the group consisting guiding fluid infusion, guiding resuscitation, preventing dehydration, preventing pulmonary edema, identifying poor contractility, managing fluids, and managing cardiac function.

17. The method of claim 10, wherein the range of assessment angles comprises angles corresponding to at least one target assessment area selected from the group consisting of a descending aorta view, a left ventricle view, and a left ventricular outflow tract view.

18. The method of claim 10, further comprising maintaining the assessment device within a patient for greater than 48 hours.

19. The method of claim 10, further comprising maintaining the assessment device within a patient for greater than 5 days.

20. The method of claim 10, further comprising displaying one or both of the Doppler assessment data point on a display device or a Doppler assessment corresponding to the Doppler assessment data point.
